(19) United States
(12) Patent Application Publication
(10) Pub. No.: US 2006/0073202 A1
(43) Pub. Date: Apr. 6, 2006

(54) Dual component medicament delivery system

(76) Inventors: Jose G. Rocca, Miami, FL (US); Khalid Shah, Davie, FL (US)

Correspondence Address: Kos Pharmaceuticals, Inc.;25th Floor, 1001 Brickell Bay Drive, Miami, FL, 33131, US

(21) Appl. No.: 10/958,948

(22) Filed: Oct. 5, 2004

(51) Int. Cl. A61K 9/46

(52) U.S. Cl. 424/466

(57) ABSTRACT

A medicament delivery system is disclosed. The system comprises a descending formulation for delivering the medicament and an ascending formulation to protect a patient being treated with the medicament from the side effects thereof.

### BACKGROUND OF THE INVENTION

1. Field of the Invention

This invention relates to a medicament delivery system comprising (a) a descending formulation for delivering the medicament to the lower stomach area of a patient being treated by the medicament or in need thereof; and, (b) an ascending formulation for providing a protective barrier to the esophagus area and the upper stomach area of the patient, which protects these areas from the side effects of the medicament.

2. Description of the Related Art

One approach to addressing the problem of gastric reflux or other gastrointestinal (“GI”) problems resulting from the administration of caustic medicaments comprises the administration of a preparation that forms a gelatinous foam or raft which floats on the stomach contents. The gelatinous foam or raft may be administered in conjunction with the medicament to produce a foam-containing medicament. The foam-containing medicament, e.g. an antacid, precedes the stomach contents into the esophagus when reflux occurs and helps to protect the mucosa from further irritation. The gelatinous foam or raft layer may be formed by the combination of an acid insoluble gelatinous material entrapping carbon dioxide (“CO₂”) gas.

Antacid compositions which contain a gel-forming agent and an acid neutralizing agent are known (cf. N. Washington et al. Int. J. Pharm. 27, 1985, pp. 279-286 and N. Washington et al. Int. J. Pharm. 28, 1986, pp. 139-143). The gel-forming agent in these known compositions is alginic acid and they further contain sodium bicarbonate and usually at least one other acid neutralizing agent as well.

Typically when alginate-based calcium carbonate/sodium bicarbonate containing rafting antacids come into contact with the acid contents of the stomach, the water insoluble calcium carbonate dissolves, liberating calcium ions. The liberated calcium ions react with the alginate to form a gelatinous mass of calcium alginate. Much of the carbon dioxide liberated from the sodium bicarbonate becomes trapped in the mass causing it to rise as a “raft” of neutral gel that effectively impedes reflux. In severe cases this neutral gel itself may be refluxed into the esophagus, where it is believed to protect the inflamed mucosa, allowing healing to take place and preventing further inflammation or ulceration.

Several formulations have been developed in order to produce an antacid suspension with rafting properties, combining antacid placement in the upper stomach/lower esophagus with prolonged buffering ability to ensure that refluxed material is close to neutral pH. Heretofore, known preparations used to create the aforementioned “rafts” comprise sodium bicarbonate or magnesium carbonate combined with either a solid composition or a liquid suspension of alginic acid or its sodium salt. Exemplary of such prior art preparations include the product sold under the trade name GAVISCON® (Marion Laboratories) and compositions described in U.S. Pat. No. 4,140,760. Each GAVISCON® tablet contains 200 mg of alginic acid and 40 mg of magnesium carbonate.

In addition, certain compositions for raft forming antacids are disclosed in the following U.S. patents: U.S. Pat. No. 5,112,813 describes a process for producing a viscosity-stable rafting antacid composition using potassium bicarbonate, magnesium carbonate, aluminum hydroxide, magnesium alginate and xanthan gum as a stabilizer.

U.S. Pat. No. 5,068,109 describes an antacid composition with floating properties containing potassium bicarbonate, magnesium carbonate and pectin.

U.S. Pat. No. 5,036,057 describes a method of treating gastroesophageal reflux using a composition with rafting properties containing calcium carbonate, sodium bicarbonate, aluminum hydroxide or magnesium carbonate and sodium alginate.

U.S. Pat. No. 4,869,902 describes a pharmaceutical composition for treatment of reflux using a composition with rafting properties containing calcium carbonate, sodium bicarbonate, aluminum hydroxide or magnesium carbonate and sodium alginate.

U.S. Pat. No. 4,744,986 describes a pharmaceutical composition for treatment of reflux esophagitis with rafting properties containing potassium bicarbonate, magnesium carbonate, aluminum hydroxide, stabilizer and magnesium alginate.

U.S. Pat. No. 4,613,497 describes a dry, water-foamable pharmaceutical composition for a gastric antacid material producing rafting containing carrageenan, sodium bicarbonate, tartaric acid, calcium carbonate, aluminum hydroxide and magnesium hydroxide.

U.S. Pat. No. 4,465,667 describes a processing for preparing gastric acid neutralizing agents having rafting properties containing magnesium carbonate, hexitol stabilized aluminum hydroxide, and hydrogenated glucose polymers.

U.S. Pat. No. 4,012,333 describes preparation of gels of beta-1,3-glucan-type polysaccaride by exposure to CO₂ gas.

U.S. Pat. No. 4,140,760 describes a pharmaceutical composition for treatment of reflux esophagitis with rafting properties containing potassium bicarbonate, magnesium carbonate and sodium alginate.

U.S. Pat. No. 2,774,710 describes an antacid preparation combining known antacids and a composition producing a protective layer containing a guar gum.

Raft formulations have been utilized with antacid formulations and other formulations that require dissolution of the medicament in the gastric juices of the stomach. However, many medicaments require dissolution in locations other than the stomach. In addition, many medicaments exhibit gastric irritation that requires patient compliance to ameliorate, such as dietary restrictions and vertical alignment restrictions. For example, patients who utilize sodium alendronate are instructed not to lie down for thirty minutes after administration. To date, no formulations have combined these medicaments with raft formulations. What is needed is a formulation or vehicle that addresses GI irritation while concurrently providing oral delivery of a medicament to a patient for systemic treatment of a disease state.

### SUMMARY OF THE INVENTION

This invention relates to a medicament delivery system, and, more particularly, to a delivery system comprising (a) a descending formulation comprising the medicament for delivering the medicament to a patient and (b) an ascending formulation for delivering a protective barrier to the patient.

### DETAILED DESCRIPTION OF THE INVENTION

As used herein, the following terms have the indicated meaning:
(1) the term “descending formulation” means a composition, combination or mixture comprising the active pharmacological ingredient, drug or medicament and at least one agent which renders the formulation more dense than the gastric juices contained in the lower stomach area of a patient, e.g. a human or other mammal, being treated or in need of the medicament. The descending formula will release the active pharmacological ingredient, drug or medicament in the GI tract.

(2) the term “ascending formulation” means a composition, combination or mixture comprising at least one protective agent which delivers a protective barrier, e.g. a film or coat, to the patient, which protects areas of the patient, e.g. the lower esophagus, the upper stomach, from the effects of the medicament, its irritants, and/or its side-effects, i.e., gastric irritation;
(3) the term “patient” shall mean any mammal.

There are many drugs whose side effects or irritants cause gastroesophageal irritation or other harmful effects when taken by a patient in need thereof. Some of these drugs include the following categories: bisphosphonates, e.g., alendronate, risendonate, etodronate, etc.; antiarrythmics, e.g. quinidine, procainamide, mexitil, quinine etc.; NSAIDs, e.g. naproxen, ibuprofen, etc.; corticosteroids, e.g. cortisone; narcotic analgesics, e.g. propoxyphene, anti alzheimer's drugs e.g. donepezil; niacin analogs, e.g. acipimox or orally administered antineoplastic agents where irritation to the gastrointestinal (“GI”) tract must be prevented. Some of the drugs can cause gastric irritation as a result of direct contact with the area, such as the product alendronate sodium sold under the trademark FOSAMAX, or by stimulation of gastric secretion leading to irritation or ulceration, such as the product donepezil hydrochloride sold under the trademark ARICEPT®. In the case of alendronate sodium, the dosage regime instructs patients to avoid lying down for at least 30 minutes following administration to facilitate delivery of the drug to the systemic system and minimize potential esophageal irritation. In addition, patients are instructed not to take alendronate sodium at bedtime or before arising for the day. Side effects of this particular drug have been reported and have included esophagitis, esophageal ulcers and erosions requiring hospitalization.

The medicament selected is combined with a suitable descending agent to form a descending formulation. A suitable descending agent is one that will give the descending formulation a density greater than the density of the gastric juices contained in the stomach of a patient. The density of the gastric juices in the stomach of a human is typically less than 1 g/ml. The density of conventional medicament tablets is usually about 1.0 to about 1.5 g/ml. Most components of the pellets and tablets have densities in this range or less. Some components are heavier but in practice are always used with other, lighter, components such that the final density of the pellets or tablets is within the conventional range.

The descending agent is preferably in particulate form. It generally has a density of at least about 2.5 g/ml preferably at least about 3.0 g/ml, more preferably at least about 3.5 g/ml, generally more than about 4.0 or sometimes more than about 5.0 g/ml. Usually the density is less than about 10 g/ml and often need be no more than about 6.0 g/ml.

Examples of suitable descending agents are shown in the following table that shows their densities in g/cm3.

| Compound | Density |
|---|---|
| Magnesium trisilicate | 3.2 |
| Magnesium oxide | 3.6 |
| Aluminum oxide | 4.0 |
| Titanium dioxide | 3.9-4.2 |
| Barium sulphate | 4.5 |
| Ferric oxide | 4.5 |
| Aluminum calcium silicate | 3.0 |
| Aluminum sodium silicate | 2.6-3.3 |
| Tricalcium phosphate | 3.1 |
| Magnesium carbonate | 3.0 |
| Calcium silicate | 2.9-3.3 |
| Calcium carbonate | 2.7-2.8 |
| Ferrum Redactum | 4.26 |
| Titanium hydroxide | 2.0-3.0 |

The most suitable are barium sulphate, ferric oxide, ferrum redactum, magnesium oxide, titanium dioxide and hydroxide. The preferred weighting agents are barium sulphate and calcium dibasic phosphate.

The amount of descending agent is selected to give the desired density and this in turn depends, in part, on the packing density and thus the particle size and shape of the descending agent and the other components. Often the amount of the descending agent is above about 50% or about 60% by weight, usually below about 90% or about 95% by weight, based on the dry weight of the unit, with particularly good results often being achieved with values of around about 70% or about 75% by weight up to about 90% by weight.

The resultant mixture of medicament and descending agent is combined into particles using conventional techniques, e.g., wet/dry granulation, extrusion/spheronization, etc. For instance, granules of the medicament or active ingredient can be used as cores and then coating the particulate cores with the dense material, i.e., the descending agent. If a controlled release formulation is desired, the coating can additionally contain a controlled release binder, e.g. acrylic polymers, cellulose derivatives, etc. Alternatively, the dense material particles, i.e. the descending agent and optional binder may be formed into heavy cores that are then coated with the active ingredient or drug and with the optional binder.

Effective quantities of the active ingredient contained in the descending formulations, destined to be combined or mixed i.e. associated with an ascending formulation, are administered to a patient orally (i.e. capsules or tablets). Some medicaments of the descending formulation are in their free base. The free base while effective themselves, may be formulated and administered in the form of their pharmaceutically acceptable addition salts for purposes of stability, convenience of crystallization, increased solubility and the like.

Acids useful for preparing the pharmaceutically acceptable acid addition salts include inorganic acids such as hydrochloric, hydrobromic, sulfuric, nitric, phosphoric and perchloric acids, as well as organic acids such as tartaric, citric, acetic, succinic, maleic, fumaric and oxalic acids.

The weight of each descending formulation is generally below about 3 g, typically in the range of about 0.3 g or about 0.5 g up to about 2 g. The amount of medicament in each dosage form is usually less than about 1 g and often less than about 0.25 g. It can be as low as about 0.0001 g, for instance if it is a prostaglandin or other material, e.g. low therapeutic drug dosage examples are warfarin, hydrochlorothiazide etc., that are therapeutically active at very low dosages. Low therapeutic doses may be at least about 0.01 g and are usually at least about 0.1 g.

The amount of the descending agent is selected to give the desired density to the descending formulation and this in turn depends upon the packing density and the particle size and shape of the descending agent and the other components of the descending formulation.

Typically, the amount of the descending agent is above about 50% or about 60% by weight, usually below about 90% or about 95% by weight, based on the dry weight of the descending formulation, with particularly good results often being achieved with values of around 70% or about 75% by weight to about 90% by weight. The particle size of the descending formulation ranges from about 25 microns to about 2 mm.

If a binder is employed in the descending formulation, the binder (including its manner of application) determines the rate of release of the active ingredient from within each unit. For instance, if the binder is readily soluble in gastric juices the unit will disintegrate rapidly upon entry to the stomach, giving substantially immediate release of all its active ingredient. If the binder (which may be a matrix binder or a coating around the unit) is a gastric controlled release binder it will not permit disintegration upon entry to the stomach but will instead permit permeation, at a pre-selected time and rate, of active ingredient into the gastric juices. This makes the active ingredient available within the stomach and also within the upper intestine into which the stomach fluids are expelled. If the binder is composed of an enteric component it prevents release of active ingredient within the stomach but instead permits release only at the higher pH conditions that prevail in the upper intestine. Thus, enteric units do not release active ingredient into the stomach.

For controlled release of the active ingredient of the descending formulation, the formulation may include as a gastric controlled release binder that will permit controlled release of the active ingredient from the unit while in the stomach, and, optionally, that will additionally provide subsequent release within the intestine. The gastric controlled release binder may consist of a matrix binder which bonds the other components of the unit together in such as to control release of the medicament or the gastric controlled release binder may consist of a coating around the unit. It may be unnecessary to have any additional binder, or there may be a conventional matrix binder.

Known material for use as conventional matrix binders are generally polymers. They may be natural polymers (e.g. starch) or synthetic polymers or derivatives (e.g. cellulose). The preferred material is cellulose based, which may necessitate other inactive ingredients to aid in the manufacturing process. Normally the matrix binder is relatively-insoluble but permeable in water.

The gastric controlled release binder, that is present as either a coating or as a matrix binder or as both, may be selected from any of the conventional controlled release binders that will permit controlled release of the medicament at the desired time and rate. It can, for instance, be formulated to permit gradual release only after a predetermined residence time in the stomach. Generally, however, it is formulated in a conventional manner to permit gradual, but substantially immediate, release from within about 15 to about 45 minutes after administration to the stomach. Often the binder is such as to permit release to be sustained for at least about three hours within the stomach, and may be such as to permit release to continue after the unit is expelled from the stomach.

Such binders are well known and generally comprise hydrophobic acrylic polymers or cellulose derivatives, vinyl polymers and other high molecular weight natural polymer derivatives or synthetic polymers. Preferred enteric polymers include, but are not limited to, anionic polymers of methacrylic acid and methacrylates; copolymers of acrylate and methacrylates (with quaternary ammonium group as a functional group); or cationic polymers with dimethyl-aminoethyl ammonium functional groups. The first and last groups of polymers may be used either alone or with combinations with any of the others. Other examples of enteric polymers are cellulose acetate phthalate, cellulose acetate succinate, styrol maleic acid co-polymers, dimethylaminoethylacrylate/ethylmethacrylate copolymers or dimethylaminoethyl methacrylates.

The controlled release coating or other binder may optionally comprise other pharmaceutically acceptable materials which improve the properties of the coating or binder, such as plasticizers, anti-adhesives, diffusion-accelerating or retarding substances, colourants, opacifiers or fillers. For example a plasticizer known to work well with ethyl cellulose is acetyltributyl citrate.

Any controlled release coating is typically about 10 um to about 100 um thick. The film may be applied by spraying the binder dissolved or dispersed in a solvent system onto a moving bed of the units. Most widely used methods are the fluidized bed and pan coating systems, the preferred method being the fluidized bed method.

Reference with respect to the above is made to U.S. Pat. No. 5,374,430, which has been incorporated by reference hereinto in its entirety.

As indicated previously, the descending formulation is combined or associated with an ascending formulation. By “combined” or “associated” is meant that the two formulations are contained together, as layered one atop the other in a selected configuration, or encapsulated together or entrapped by a polymeric matrix, mixed together as powders, combined together as beads or pellets, etc. However, in all embodiments, the descending formulation remains a discreet and separate component in the finished product. For example, a descending formulation comprising barium sulphate and sodium alendronate is co-extruded to produce particles comprising an average diameter of about 2.5 microns. These particles are added to the ingredients necessary to form the raft along with any other excipients necessary to formulation oral dosage formulations, for example tablets or capsules. In an alternative embodiment, particles of the ascending formulation and particles of the descending formulation are formulated into a suspension.

The ascending formulation, also known as the raft layer, comprises materials adapted to float on gastrointestinal fluids contained in the stomach. Raft layer compositions are well known in the art. In this regard, reference is made to U.S. Pat. Nos. 4,140,760; 5,360,793; 5,112,813; 5,068,109; 5,036,057; 4,869,902; 4,744,986; 4,613,497; 4,465,667; 4,012,333; 4,140,760 and 2,774,410, all of which are incorporated hereinto by reference in their entirety. The present invention improves upon the prior raft layer technology by enabling the use of medicaments that dissolve in parts of the body other than the stomach or that cause irritation to the stomach.

The most common raft layer generating means comprises (a) a suitable antacid which can serve as a CO₂ source; (b) a gelatinous foam or raft layer producing gel agent and optionally (c) a CO₂ source material in supplement to (a), above. The antacids typically comprise basic metal salts. Other CO₂ generating materials may be acid substances and carbonates or bicarbonates that react in the presence of water releasing CO₂.

Examples of metals known to form basic salts are alkali or alkaline earth metals and aluminum. The most commonly employed aluminum salts are the hydroxide, carbonate or phosphate. Examples of alkaline earth metals such as calcium are known, the use of calcium carbonate as an antacid either alone or in combination with other metal salts, such as magnesium carbonate and magnesium hydroxide is known. The use of alkali metals, such as sodium carbonate, bicarbonate is known in antacid formulations.

The gel forming agent is typically a source of an alginic acid salt upon entry of the ascending formulation in the body of the patient. For example, when a formulation of an alginate-based calcium carbonate/sodium bicarbonate formulation comes into contact with the acid contents of the stomach of a patient being treated the water insoluble calcium carbonate dissolves, liberating calcium ions which then react with the alginate to form a gelatinous mass of calcium alginate. Much of the carbon dioxide liberated from the calcium carbonate and from the sodium bicarbonate becomes trapped in the mass causing it to rise as a “raft” of neutral gel which forms a protective barrier or coat on the lower esophagus area and the upper stomach area of the patient. This protective barrier film or coat coats the surfaces of these areas protecting them from the gastric irritation side effects of the medicament destined to be transported thereto and to the lower stomach of the patient.

Another gel forming agent is xanthan gum which forms a gelatinous mass with hexitol stabilized aluminum medroxide, as reported in U.S. Pat. No. 5,360,793, which has been incorporated hereinto by reference in its entirety.

Other raft generating means comprise (a) a substance which is soluble in water at a neutral pH or alkaline pH but is capable of forming a cohesive gel at an acid pH, (b) a substance which is capable of acting as a buffer and is capable of being captured in the cohesive gel structure formed at an acid pH and (c) one or more acid neutralizing agents.

Examples of substances that are soluble in water at a neutral pH or alkaline pH but are capable of forming a cohesive gel at an acid pH include pectin, alginate, carrageenan or a cellulose-derivative such as a carboxymethylcellulose. Examples of the buffering substance include proteinaceous substances, such as casein, caseinate and milk powder. Finally, the acid neutralizing agents include a CO₂ source material.

A preferred ascending formulation comprises sodium alginate (100 g), sodium bicarbonate (80 g), calcium carbonate (18 g).

As indicated, the ascending formulation is associated with the descending formulation, e.g., typically, mixed together in a capsule or housing system. When such mixture or capsule is administered orally to the patient, the filled capsule is dissolved, thereby releasing the ingredients therein. The components of the ascending formulation delivers the protective barrier to the lower esophagus and upper stomach areas of the body of the patient by the raft layer mechanism while, concurrently therewith, the descending formulation particles descend to the lower stomach area delivering the active ingredient or medicament. As indicated, the above-description is exemplary only and the two formulations can be combined or associated in any preferred manner to both protect the patient from the side effects of a drug while delivering the drug itself to the area of the body of the patient where such drug is efficacious.

For the purpose of oral therapeutic administration, the medicaments contained in the descending formulation destined to be combined with the ascending formulation may be incorporated with excipients and used in the form of tablets, troches, capsules, caplets, elixirs, suspensions, syrups, wafers, chewing gum and the like. The amount of active compound in such compositions is such that a therapeutic dosage will be delivered.

The tablets, pills, capsules, troches and the like may also contain the following ingredients; a binder such as microcrystalline cellulose, gum tragacanth or gelatin; an excipient such as starch or lactose; a disintegrating agent such as alginic acid, primogel, corn starch and the like; a lubricant such as magnesium stearate or Sterotex; a glidant such as colloidal silicon dioxide; and a sweetening agent such as sucrose or sacchrin; and a flavoring agent such as peppermint, methyl salicylate, or orange flavoring. When the dosage unit is a capsule. It may contain, in addition to materials of the above type, a liquid carrier such as fatty oil. Other dosage unit forms may contain other various materials that modify the physical form of the dosage unit, for example, as coatings. Thus, tablets or pills may be coated with sugar, shellac or other enteric coating agents. A syrup may contain, in addition to the active compounds, sucrose as a sweetening agent and certain preservatives, dyes and colorings and flavors. Materials used in preparing these various compositions should be pharmaceutically pure and non-toxic in the amounts used.

We claim:

1. A raft layer generating pharmaceutical composition comprising a material adapted to float on gastrointestinal fluids contained in the stomach, wherein the improvement comprises the addition of a descending formulation having a medicament and a descending agent.

2. The raft layer generating pharmaceutical composition of claim 1, wherein said descending formulation has a density greater than that of the gastric juices of said stomach.

3. The raft layer generating pharmaceutical composition of claim 1, wherein said descending agent has a density of at least about 2.5 g/ml.

4. The raft layer generating pharmaceutical composition of claim 2, wherein said descending formulation is a unit having a descending agent and a medicament.

5. The raft layer generating pharmaceutical composition of claim 4, wherein said unit comprises a medicament bound to said descending agent.

6. The raft layer generating pharmaceutical composition of claim 4, wherein said unit has A descending agent coated with a medicament.

7. A medicament delivery system, which comprises:
(a) a descending formulation for delivering the medicament to the lower stomach area of a patient being treated by; and
(b) an ascending formulation associated with said descending formulation for providing a protective barrier to the esophagus area and the upper stomach surface area of said patient to protect said areas from the side effects of the medicament.

8. The medicament delivery system as defined in claim 7, wherein said descending formulation has a density greater than that of the gastric juices of said lower stomach area.

9. The medicament delivery system as defined in claim 8, wherein said descending formulation comprises a descending agent and a medicament.

10. The medicament delivery system as defined in claim 9, wherein said descending agent has a density of at least about 2.5 g/ml.

11. The medicament delivery system as defined in claim 9, wherein said particle comprises a medicament coated with a descending agent.

12. The medicament delivery system as defined in claim 9, wherein said particle comprises a descending agent coated with a medicament.

13. The medicament delivery system as defined in claim 7, wherein said ascending formulation comprises a raft layer generating means.

14. The medicament delivery system as defined in claim 13, wherein said raft layer generating means comprises a composition containing a source of CO₂.

15. A method of delivering a medicament to a patient to avoid gastric irritation of the patient due to side effects of the medicament which comprises incorporating the medicament into a descending formulation associated with a raft layer generating pharmaceutical composition.

16. The method as defined in claim 15, wherein said descending formulation has a density greater than that of the gastric juices of said stomach area.

17. The method as defined in claim 16, wherein said descending formulation comprises a particle having a descending agent and a medicament.

18. The method as defined in claim 17, wherein said descending agent has a density of at least about 2.5 g/ml.

19. The method as defined in claim 17, wherein said particle comprises a medicament coated with a descending agent.

20. The method as defined in claim 17, wherein said particle comprises a descending agent coated with a medicament.

21. A method of reducing the gastric irritation of a medicament administered to a patient, which comprises forming a descending formulation having said medicament and associating said descending formulation with a raft layer generating pharmaceutical composition.

22. The method as defined in claim 20, wherein said descending formulation has a density greater than that of the gastric juices of said lower stomach area.

23. The method as defined in claim 21, wherein said descending formulation comprises a particle having a descending agent and a medicament.

24. The method as defined in claim 22, wherein said descending agent has a density of at least about 2.5 g/ml.

25. The method as defined in claim 22, wherein said particle has a medicament coated with a descending agent.

26. The methods defined in claim 22, wherein said particle has a descending agent coated with a medicament.
